(19) United States
(12) Patent Application Publication
(10) Pub. No.: US 2007/0081952 A1
(43) Pub. Date: Apr. 12, 2007

(54) Composition for the treatment of bad breath

(76) Inventor: Christiaan Cardon, Brugge (BE)

Correspondence Address: DENNISON, SCHULTZ & MACDONALD, 1727 KING STREET, SUITE 105, ALEXANDRIA, VA, 22314, US

(21) Appl. No.: 10/578,621

(22) PCT Filed: Sep. 29, 2004
(86) PCT No.: PCT/IB04/03637

§ 371 Date: Oct. 24, 2006

(30) Foreign Application Priority Data
Sep. 29, 2003 (FR) 03/11384

(51) Int. Cl. A61K 8/96

(52) U.S. Cl. 424/50

(57) ABSTRACT

A composition suitable for treating bad breath and including a carrier material, at least one enzyme sulphite oxidase, an enzyme for breaking down the starch and/or cellulose present in the oral cavity to give glucose, an enzyme oxidoreductase, a source of halogen or pseudohalogen ions, and an enzyme peroxidase.

The object of the present invention is a composition comprising an enzyme mixture for the treatment of bad breath in man or animals.

Bad breath affects many people, and poses a serious problem, both for those suffering from bad breath and for relatives and friends as it generates mutual discomfort and may put a strain on a person's social life.

In the same way, bad breath in pets is one of the main reasons causing owners to maltreat their dogs or cats. The fact that pets live in the owner's house or flat means that having an animal with bad breath may be a source of discomfort and cause a considerable problem for the owner.

Bad breath is a problem frequently encountered in animals, in particular in pets with a small mouth, as this is not regularly aired and badly oxygenated. Under these anaerobic conditions, there is a majority of bacteria causing putrefaction.

Studies carried out to date have shown that the main compounds responsible for bad breath are volatile sulphur compounds such as dihydrogen sulphide (H2S), methyl mercapatan (CH3SH) and dimethyl sulphide (CH3SCH3). These compounds are formed by the breakdown of sulphur-containing proteins in the oral cavity by anaerobic bacteria.

Numerous compositions intended to fight bad breath have already been described.

Preparations containing chlorhexidine are commonly used to fight the bacteria causing protein breakdown in the mouth.

Thus, patent EP 920857 describes an oral composition including a) chlorhexidine digluconate b) cetyl pyridinium chloride and c) a pharmaceutically acceptable salt or a Zn²⁺ and/or Cu²⁺ compound.

Such preparations have side effects such as staining of the teeth or a change of the taste in the mouth etc Preparations containing sodium chlorite have also been proposed as this provides a source of oxygen which oxidises volatile sulphur compounds into odourless compounds. Hence, U.S. Pat. No. 6,325,997 patent describes a preparation containing sodium chlorite (NaClO₂) and a metal ion such as zinc which may form a complex with sulphur compounds.

The disadvantage of preparations containing sodium chlorite is the very poor stability of products providing oxygen. An oxygenated solution loses its oxidising activity during time (shelf-life of only 2 to 3 months for hydrogen peroxide).

Preparations containing zinc have been proposed as this metal forms an insoluble composition with volatile sulphur compounds. These preparations are taken orally and are digested. Hence application WO99/17735 describes a composition comprising a chelate containing a metal ion, preferably a zinc ion and an amino-acid, preferably glycine, which releases the chelate in a controlled manner in the oral cavity of subjects with bad breath.

However there are numerous contraindications to preparations containing zinc: cases of hypersensitivity to zinc, possible interactions with drugs and nutritional supplements, contraindication in. the event of pregnancy, breast-feeding, side effects (gastric discomfort, nausea, vomiting, headache, drowsiness, metallic taste in the mouth etc . . . ) Products containing enzymes intended to fight against bad breath have also been developed. U.S. Pat. No. 4,564,519 describes a chewable di-enzymatic toothpaste containing both glucose and glucose oxidase to produce hydrogen peroxide during mastication of the toothpaste, and also a thiocyanate salt and a lactoperoxidase to interact with the hydrogen peroxide and produce hypothiocyanate which is a bacterial inhibitor. U.S. Pat. No. 5,336,494 describes an enzymatically coated chewable product which has anti-bacterial effects in the oral cavity when it is chewed by activation of the enzymatic system contained in the coating. The enzymatic coating contains an oxidisable substrate such as beta-D-glucose or example, a specific oxidoreductase enzyme of this substrate such as glucose oxidase, in order to produce hydrogen peroxide during chewing of the aforesaid coated product, which may also contain a peroxidase enzyme such as lactoperoxidase for example and an alkaline metal salt (such as thiocyanate for example ) in order to interact with hydrogen peroxide and produce an anionic oxidised bacterial inhibitor.

Patent EP658096 describes an orally chewable antimicrobial product for pets, comprising a carrier material, at least one oxidoreductase (such as glucose oxidase, sulphite oxidase), at least one substrate for the oxidoreductase (such as D-glucose) and a catalase (such as that derived by fermentation of Aspergillus niger) making it possible to control the production of hydrogen peroxide. The product may also contain an enzyme peroxidase (lactoperoxidase for example) and a source of halide ions.

However, it is always necessary to use a substrate for the oxidoreductase and this has disadvantages in as far as stability is concerned both during manufacture of the product, and for the finished product.

The object of the present invention is a suitable composition for the treatment of bad breath comprising:
A carrier material,
A sulphite oxidase enzyme,
At least one enzyme capable of breaking down the glucose, starch and/or the cellulose present in the oral cavity,
An oxidoreductase enzyme,
A source of halide or pseudohalide ions,
A peroxidase enzyme.

A carrier material is any material that makes the composition, object of this invention, agreeable for the oral cavity of animals or man. Examples of carrier materials that may be used are dental paste, collagen or any non-toxic product remaining for the necessary time to obtain a reaction in the oral cavity, etc . . . .

The carrier material may remain in the oral cavity for a period of from approximately 5 seconds to approximately 1 h. The quantity of carrier material varies between approximately 1 % to approximately 99% by weight of the total weight of the composition of the invention.

The sulphite oxidase enzyme makes it possible to transform the volatile sulphur compounds present in the oral cavity and responsible for bad breath, such as dihydrogen sulphide (H₂S), methyl mercaptan (CH₃SH) and dimethyl sulphide (CH3SCH3) into odourless compounds (that is sulphone (SO₂) and dimethylsulphone (CH₃SO₂CH₃)).

The quantity of the sulphite oxidase varies from approximately 0.2 IU to approximately 2000 IU.

The term “IU” means “International Unit” and indicates the quantity of enzyme required to catalyse 1 micromole of compound per unit at a pH of 7.0 and a temperature of 25° C.

The enzyme making it possible to break down the starch and/or cellulose present in the oral cavity into glucose is chosen from the group composed of amylase, cellulase, glucoamylase and their mixtures.

The quantity of enzyme making it possible to breakdown starch and/or cellulose into glucose ranges from approximately 0.05% to approximately 30% by weight compared to the total weight of the composition of the invention.

The starch and/or cellulose present in the oral cavity come from food residues. As an example, the daily diet of pets, in particular dogs, is mainly composed of cereals containing starch. In order to utilise starch for its energy value, a specific enzyme must react to release the glucose: amylase or glucoamylase. The saliva of dogs (unlike that of man) does not contain amylase (amylase is only found in dog intestines).

The use of an amylase, cellulase and/or glucoamylase enzyme has the advantage that:
Starch remaining between the teeth (in particular the teeth of dogs) is then broken down into glucose particles which are ready to be used as substrate for the “glucose oxidase”, “lactoperoxidase” reaction.
The starch is not usable as a substrate for the formation of dental plaque (if not the starch remains in the dog's mouth and remains a substrate for bacteria).

The oxidoreductase enzyme makes it possible to oxidise glucose particles into glucuronate and hydrogen peroxide.

The oxidoreductase enzyme is selected from the group comprising glucose oxidase, galactose oxidase, glycolate oxidase, aldehyde oxidase, lactate oxidase, xanthine oxidase, L-amino-acid oxidase, D-amino-acid oxidase, monophosphate oxidase, hexose oxidase, xylitol oxidase, pyranose oxidase, alcohol oxidase and their mixtures.

The quantity of oxidoreductase varies from approximately 0.2 IU to approximately 2000 IU.

A suitable glucose oxidase enzyme is for example that derived from Aspergillus sp. or a strain of Aspergillus niger or a strain of Cladosporium sp., in particular Cladosporium oxysporum.

An L-amino-acid oxidase enzyme may be appropriate for example that described in WO94/25574 or that derived from Trichoderma sp. such as Trichoderma harzianum, or Trichoderma viride.

A suitable hexose oxidase enzyme is for example that derived from red algae Chondrus crispus or Iridophycus flaccidum. Hexose oxidases of the red alga Chondrus crispus (best known by its common name, Irish moss) (Sullivan and Ikawa (1973), Biochim Biophys. Acts, 309, p. 11-22; Ikawa (1982), Meth. In Enzymol. 89, carbohydrate metabolism part D, 145-149) oxidise a broad spectrum of carbohydrates including D-glucose, D-glucose 6-phasphate, D galactose, maltose, cellobiose, lactose, D-mannose, 2-deoxy-D-glucose, 2˜-deoxy-D-galactose, D-fucase and D-xylose.) Hexose oxidases of the red alga Iridophycus flaccidum also oxidise different mono- and disaccharides (Bean and Hassid (1956), J. Biol. Chem., 218, p. 425; Rand et al. (1972, J. of Food Science 37, p. 698-710).

The enzyme xylitol oxidase which may be suitable is for example that described in JP80892242 which oxidises xylitol, D-sorbitol, D-galactitol, D-mannitol and D-arabinitol in the presence of oxygen. A xylitol oxidase may be obtained from strains of Streptomyces sp. (e.g. [Streptomyces] IKD472, FERM P-14339).

The source of halide or pseudohalide ions is selected in the group composed of potassium thiocyanate, sodium thiocyanate, ammonium thiocyanate, other thiocyanate salts, potassium iodide, other iodide salts, sodium chloride, other chloride salts and their mixtures.

The quantity of halide or pseudohalide ions varies from approximately 0.0001 mol/g to approximately 0.1 mol/g of carrier material.

The peroxidase enzyme is selected from the group comprising lactoperoxidase, superoxide dismutase, myeloperoxidase, chloroperoxidase, horseradish peroxidase, saliva peroxidase and their mixtures.

The quantity of peroxidase enzyme varies from approximately 0.1 lU/g to approximately 100 lU/g of carrier material.

The peroxidase enzyme and the source of halide or pseudohalide ions interact with hydrogen peroxide to produce an antibacterial agent hypothiocyanate (OSCN⁻/HOSCN). Lactoperoxidase-is an enzyme naturally present in saliva. However additional peroxidases such as those mentioned above may be added, and in particular superoxide dismutase which has a much faster action than lactoperoxidase.

According to an advantageous method for the preparation of the invention, the composition also includes an agent stimulating salivation, chosen in particular in the group of saturated or unsaturated emulsifiers, acidifiers and their mixtures.

The following may be mentioned as emulsifiers: alkyl aryl sulphonates, alkyl sulphates, sulphonated amides and amines, sulphated and sulphonated esters and ethers, alkyl sulphonates, polyethoxylated esters, mono- and diglycerides, diacetyl tartaric monoglyceride esters, polyglycerol esters, sorbitan and ethoxylate esters, lactyl esters, phospholipids such as lecithin, sorbitan polyoxyethylene esters, propylene glycols esters, esters of sucrose and their mixtures. More particularly, sorbitol may be mentioned as a typical emulsifier.

The following may be mentioned as- examples of acidifiers: citric acid, malic acid, tartarate, sodium chloride, potassium chloride and their mixtures. Citric acid is a particularly suitable acidifier.

The agent stimulating salivation facilitates the reaction transforming starch into glucose by ensuring that as much starch is impregnated with amylases as possible. The saliva, which is already present in larger quantities, will be used as carrier in the oral cavity.

According to another advantageous method of preparation of the invention, the composition includes a buffering agent giving the composition a pH of from approximately 4 to 8, and preferably from approximately 5.4 to 6.5.

Suitable buffering agents include monobasic potassium phosphate, dibasic potassium phosphate, monobasic sodium phosphate, dibasic sodium phosphate, sodium bicarbonate, sodium hydroxide, potassium hydroxide, citric acid, benzoic acid, malic acid etc.

The maintenance of a pH between 4 and 8, preferably between 5.4 and 6.5 increases the concentration of the antibacterial agent hypothiocyanate in the HOSCN/OSCN⁻ balance which is advantageous because HOSCN has been shown to be more active than OSCN⁻ against bacteria, as HOSCN is neutral and penetrates bacterial cells more easily.

According to another advantageous method of preparation of the invention, the composition includes a flavouring agent that may also be called “flavoured salivation stimulant” Typical examples are chicken flavours or fish flavours.

According to another advantageous method of preparation of the invention, the composition includes an antibacterial enzyme selected from the group comprising lysozyme, lactoferrin and their mixtures. Lysozyme makes it possible to destroy the bacterial membrane and lactoferrin makes it possible to absorb all the iron which is an essential element for the survival of pathogenic bacteria.

According to another advantageous method of preparation of the invention, the composition includes suitable vehicles and excipients for oral administration.

The corriposition of the invention may include an anticaries agent (fluorine, sodium fluoride, calcium lactate etc), an anti-plaque agent (zinc ion, sanguinarine etc.) an anti-tartar agent (pyrophosphate, polyphosphate, hexamethaphosphate salts etc . . . ), an antibacterial agent (chlorhexidine, phenoxyethanol, parabene etc . . . ), an anti-inflammatory agent (ibuprofen, meclofenamic acid etc . . . ), an inhibitor of the proteases involved in the inflammatory process (metalloprotein, serine proteinase etc.), an antiseptic agent (miconazole, acidovir etc . . . ) etc . . . .

It may also include a humectant (glycerin, propylene glycol, polyethylene glycol, sorbitol, mannitol, xylitol etc.), a thickening agent (carrageenin, methyl cellulose, silica gel (Tixosil©), Mg-Al-Silica colloid etc . . . ), an abrasive agent (calcium phosphate, silica, urea, formaldehyde, Tixosil® etc . . . ), a surfactant with an anionic (for example sodium laurylsulfate etc . . . ), cationic (cetylpyridine fluoride, cetylpyridine chloride etc . . . ), or non-ionic structure (such as ethylene oxide condensation products with propylene-glycol (products in the Pluronic® series etc . . . ), an antioxidant (vitamin A, cysteine, glutathion, coenzyme Q-10 etc . . . ), a sweetener (glucose, sucrose, lactose, acesulfame, etc . . . ), a freshener (menthol, carboxamide etc . . . ), a spice (capsicum, sweet pepper etc . . . ), a neutralizer (clove oil, lidocaine etc.), an emulsifier (polymeric compositions such as polyvinylmethylether etc . . . ), an agent facilitating adherence of the active substance to the support (natural gums etc . . . ), a preservative (hydroxymethyl, hydroxypropyl parabene etc . . . ), a whitening agent (urea peroxide, hydrogen peroxide etc . . . ), a colouring agent (methylene blue etc.), water.

The composition according to the invention is supplied as a liquid oral formulation, a solid oral formulation etc . . . .

In particular, the composition of the invention is presented for example in the form of a toothpaste, chewing strips, chewing gum, mouthwash, oral gel, dental powder, chewing tablet, chewing paste etc . . . .

The object of the present invention is also the use of a composition as described above for the treatment of bad breath in man or in animals.

The compositions of the invention do not require the use of a substrate for the oxidoreductase enzyme, which has the advantage that there is no oxidation of glucose by glucose oxidase in order to form the peroxide which, at the appropriate time will oxidise thiocyanate into oxythiocyanate.

The compositions of the invention have a prolonged stability in time with a shelf life of up to 60 months.

The following examples illustrate the invention, though they do not restrict it in any way.

### EXAMPLE 1

### Toothpaste

| | |
|---|---|
| Glycerin (humectant) | 40.000 g |
| Water (carrier) | 5.000 g |
| Tixosil 73 (thickener and abrasive) | 40.000 g |
| Dicalcium phosphate (buffer) | 5.000 g |
| Flavouring agent | 5.000 g |
| Potassium thiocyanate | 0.010 g |
| Lactoperoxidase | 0.010 g |
| Glucose oxidase (208 IU) | 0.010 g |
| Sulphite oxidase | 0.010 g |
| Glucoamylase | 0.200 g |
| Amylase | 0.010 g |
| Cellulase | 0.200 g |

### EXAMPLE 2

### Solution to be Applied on Collagen

| | |
|---|---|
| Water (carrier) | 40.000 g |
| Carrageenan (thickening agent) | 1.500 g |
| Flavouring Agent | 5.000 g |
| Potassium thiocyanate | 0.010 g |
| Lactoperoxidase | 0.010 g |
| Glucose oxidase (208 IU) | 0.010 g |
| Sulphite oxidase | 0.010 g |
| Glucoamylase | 0.200 g |
| Amylase | 0.010 g |
| Cellulase | 0.200 g |

### EXAMPLE 3

### Mouthwash

| | |
|---|---|
| Water (carrier) | 80.000 g |
| Propylene glycol (humectant) | 15.000 g |
| Flavouring agent | 4.500 g |
| Potassium thiocyanate | 0.010 g |
| Lactoperoxidase | 0.010 g |
| Glucose oxidase (208 IU) | 0.010 g |
| Sulphite oxidase | 0.010 g |
| Glucoamylase | 0.200 g |
| Amylase | 0.010 g |
| Cellulase | 0.200 g |

### EXAMPLE 4

### Chewable Paste

The percentages are percentages in weight relative to the total weight of the composition.

| | |
|---|---|
| Paste (carrier material) | 99% |
| Glucoamylase | 0.19% |
| Amylase | 0.01% |
| Glucose oxidase | 20 IU |
| Sulphite oxidase | 20 IU |
| Lactoperoxidase | 0.06% |
| Alkaline metal or salt | 0.013% |
| Lysozyme HCl | 0.06% |
| Lactoferrin | 0.06% |
| Sodium benzoate | 0.017% |
| Phosphate buffer | 0.14% |
| Alginate | 0.14% |
| Flavour | 0.1% |
| Salivation stimulant | 0.30% |

1. Composition for the treatment of bad breath, comprising:
A carrier material,
A sulphite oxidase enzyme,
At least one enzyme capable of breaking down glucose, starch and/or cellulose present in the oral cavity,
An oxidoreductase enzyme,
A source of halide or pseudohalide ions,
A peroxidase enzyme.

2. Composition according to claim 1, wherein the quantity of support material ranges from approximately 1% to approximately 99% by weight of the total weight of the composition.

3. Composition according to claim 1, wherein the quantity of sulphite oxidase ranges from approximately 0.2 IU to approximately 2000 IU.

4. Composition according to claim 1, wherein the enzyme making it possible to break down the starch and/or cellulose present in the oral cavity into glucose is chosen from the group composed of amylase, cellulase, glucoamylase and their mixtures and that the quantity of the aforesaid enzyme varies from approximately 0.05% to approximately 30% by weight compared to the total weight of the composition.

5. Composition according to claim 1, wherein the oxidoreductase enzyme is selected from the group comprising glucose oxidase, galactose oxidase, glycolate oxidase, aldehyde oxidase, lactate oxidase, xanthine oxidase, L-amino-acid oxidase, D-amino-acid oxidase, monophosphate oxidase, hexose oxidase, xylitol oxidase, pyranose oxidase, alcohol oxidase and their mixtures and wherein the quantity of the aforesaid enzyme varies from approximately 0.2 IU to approximately 2000 IU.

6. Composition according to claim 1, wherein the source of halide or pseudohalide ions is selected from the group composed of potassium thiocyanate, sodium thiocyanate, ammonium thiocyanate, other thiocyanate salts, potassium iodide, other iodide salts, sodium chloride, other chloride salts and their mixtures and that the quantity of the aforesaid source of ions varies from approximately 0.0001 mol/g to approximately 0.1 mol/g of carrier material.

7. Composition according to claim 1, wherein the peroxidase is selected from the group composed of lactoperoxidase, superoxide dismutase, myeloperoxidase, chloroperoxidase, horseradish peroxidase, saliva peroxidase and their mixtures, and that the quantity of the aforesaid enzyme varies from approximately 0.1 IU/g to approximately 100 IU/g of the carrier material.

8. Composition according to claim 1, wherein the composition also includes an agent stimulating salivation, chosen in particular from the group of saturated or unsaturated emulsifiers, acidifiers and their mixtures.

9. Composition according to claim 1, wherein it includes a buffering agent used to obtain a composition with a pH of from approximately 4 to approximately 8, preferably from approximately 5.4 to approximately 6.5.

10. Composition according to claim 1, wherein it contains an antibacterial enzymatic agent selected from the group comprising lysozyme, lactoferrin and their mixtures.

11. Composition according to claim 1, wherein it includes a flavouring agent selected from the group comprising chicken flavours, fish flavours and their mixtures.

12. Composition according to claim 1, wherein it includes suitable vehicles and excipients for oral administration.

13. Composition according to claim 1, wherein it is supplied in a liquid oral form.

14. Composition according to claim 1, wherein it is supplied in a solid oral form.

15. Composition according to claim 1, wherein the composition is presented in the form of toothpaste, chewing strips, chewing gum, mouthwash, oral gel, dental powder, chewing tablet, chewing paste.
